(19) United States
(12) Patent Application Publication
(10) Pub. No.: US 2024/0408122 A1
(43) Pub. Date: Dec. 12, 2024

(54) PHARMACEUTICAL COMPOSITION FOR PREVENTION OR TREATMENT OF METABOLIC DISEASE COMPRISING LYSOPHOSPHOLIPID

(71) Applicant: SEOUL NATIONAL UNIVERSITY R&DB FOUNDATION, Seoul (KR)

(72) Inventors: YUN HEE LEE, Seoul (KR); YOON KEUN CHO, Seoul (KR); HYEON YEONG IM, Seoul (KR); SU MIN LEE, Seoul (KR)

(21) Appl. No.: 18/700,884

(22) PCT Filed: Aug. 5, 2022
(86) PCT No.: PCT/KR2022/011626

§ 371 Date: Apr. 12, 2024

(30) Foreign Application Priority Data
Oct. 18, 2021 (KR) 10-2021-0138372

(51) Int. Cl. A61K 31/685; A61P 3/04

(52) U.S. Cl. CPC A61K 31/685; A61P 3/04

(57) ABSTRACT

A composition for the prevention or treatment of metabolic diseases, which includes lysophospholipid. The pharmaceutical composition of the present invention promotes the PKA signaling pathway and upregulates lipolysis and mitochondrial oxidative metabolism, and thus can prevent or treat a metabolic disease such as obesity or diabetes.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No. PCT/KR2022/011626 filed on Aug. 5, 2022, which claims priority to the benefit of Korean Patent Application No. 10-2021-0138372 filed in the Korean Intellectual Property Office on Oct. 18, 2021, the entire contents of which are incorporated herein by reference.

### BACKGROUND

### 1. Technical Field

The present invention relates to a pharmaceutical composition for preventing or treating metabolic diseases, which includes lysophospholipid.

### 2. Background Art

Metabolic disease is collectively called diseases occurring due to lifestyle habits such as obesity, lack of exercise, and excessive nutrition, etc., and refers to various diseases related to metabolism. Specifically, the metabolic disease includes obesity, diabetes, insulin tolerance, fatty liver, hyperlipidemia, arteriosclerosis, or complications thereof exist together. Recently, it is known that the incidence of these diseases is rapidly increasing in Korea as well, and has been significantly increased at or above the levels of the United States and Western European countries.

In recent years, there has been a lot of change in eating habits according to economic growth and lifestyle changes. In particular, the proportion of busy modern people who are overweight or obesity continues to increase due to high-calorie diets such as fast foods and a little exercise. According to the World Health Organization (WHO), it has been reported that 1 billion or more adults worldwide are overweight, and at least 3 million or more of them are clinically obese, especially 250,000 people in Europe and 2.5 million or more people worldwide have died in relation to overweight.

Overweight and obesity may increase blood pressure and cholesterol levels to cause or worsen various chronic diseases and complications thereof, such as heart disease, diabetes, arthritis, fatty liver, hyperlipidemia, cancer and the like. In addition, the overweight and obesity are known to be major factors that increase the incidence of diseases such as arteriosclerosis, hypertension, hyperlipidemia or heart disease not only in adults but also in children and adolescents. Accordingly, there is a growing need to recognize obesity as a disease and actively treat it.

### SUMMARY

An aspect of the present invention is to provide a pharmaceutical composition for preventing or treating metabolic diseases, which includes lysophospholipid.

1. A pharmaceutical composition for preventing or treating metabolic diseases, including lysophospholipid (LPL).

2. The composition according to the above 1, wherein the metabolic disease is at least one selected from the group consisting of obesity, diabetes, hyperlipidemia, hypertension, hypercholesterolemia, hyperinsulinemia, arteriosclerosis and fatty liver.

3. The composition according to the above 1, wherein the lysophospholipid is lysophosphatidyl choline (LPC) or lysophosphatidyl ethanolamine (LPE).

4. The composition according to the above 3, wherein the lysophosphatidylcholine or lysophosphatidylethanolamine is at least one selected from the group consisting of p-14:1, 16:0, 18:0, 18:1, 20:0, 20:2, 20:4, 22:5 and 22:6.

5. A health functional food for preventing or improving metabolic diseases, including lysophospholipid.

6. The health functional food according to the above 5, wherein the metabolic disease is at least one selected from the group consisting of obesity, diabetes, hyperlipidemia, hypertension, hypercholesterolemia, hyperinsulinemia, arteriosclerosis and fatty liver.

7. The health functional food according to the above 5, wherein the lysophospholipid is lysophosphatidyl choline (LPC) or lysophosphatidyl ethanolamine (LPE).

8. The health functional food according to the above 7, wherein the lysophosphatidylcholine or lysophosphatidylethanolamine is at least one selected from the group consisting of p-14:1, 16:0, 18:0, 18:1, 20:0, 20:2, 20:4, 22:5 and 22:6.

The pharmaceutical composition including lysophospholipid of an embodiment of the present invention may prevent or treat metabolic diseases such as obesity or diabetes by promoting the PKA signaling pathway and upregulating lipolysis and mitochondrial oxidative metabolism.

### BRIEF DESCRIPTION OF THE DRAWINGS

FIGS. 1A and 1B show results of immunoblot analysis of C3H10T1/2 adipocytes treated with LPC/LPE P-18:0.

FIGS. 2A to 2C show results of measuring the intracellular CAMP level and oxygen consumption rate of C3H10T1/2 adipocytes treated with LPC/LPE P-18:0.

FIGS. 3A and 3B show results of immunoblot analysis of BAT, iWAT and gWAT treated with VC or LPE in mice fed a high-fat diet (HFD).

FIG. 4 shows results of testing glucose tolerance after intraperitoneal administration of VC or LPE P-18:0 to HFD-fed mice.

FIGS. 5A to 5E show results of analyzing body weight, body composition, and adipose tissue mass after treating HFD-fed mice with VC or LPE P-18:0 (FIGS. 5A and 5B), FIGS. 5C and 5D show results of indirect calorimetry analysis, and FIG. 5E shows results of testing intraperitoneal insulin tolerance.

FIG. 6 shows results of immunoblot analysis of C3H10T1/2 adipocytes treated with LPC P-16:0.

### DETAILED DESCRIPTION

Hereinafter, an embodiment of the present invention will be described in detail.

The embodiment of the present invention may provide a pharmaceutical composition for preventing or treating metabolic diseases, which includes lysophospholipid (LPL).

The lysophospholipid refers to a substance that has a lyso form among phospholipids. Recently, the lysophospholipid is recognized as a hormone-like substance that exhibits physiological activity, and is known to act mainly through G protein-coupled receptors (GPCRs) in the cell membrane.

Types of the lysophospholipid of the present invention is not limited as long as they can have medicinal effects on metabolic diseases, and may include, for example, lysophosphatidic acid (radyl-lyso-glycerophosphate, LPA), 2,3-cyclic phosphatidic acid, 1-alkyl-2-acetyl-glycero-3-phosphate, sphingosine-1-phosphate (S1P), dihydro-sphingosine-1-phosphate, sphingosyl phosphorylcholine (lysosphingomyelin, SPC) and lysophosphatidylcholine (LPC), psychosine (PSY), or lysophosphatidylserine (LPS), and preferably lysophosphatidylcholine (LPC) or lysophosphatidylethanolamine (LPE) in terms of promoting PKA signaling pathways and upregulating lipolysis and mitochondrial oxidative metabolism.

The lysophosphatidylcholine is a molecule in which one long fatty acid is bound to sn-C1 of glycerol and phosphocholine is bound to sn-C3, and is made by hydrolyzing one acyl group in phosphatidylcholine due to phospholipase (PLA2) in vivo. Types thereof may be, for example, p-14:1, 16:0, 18:0, 18:1, 18:2, 20:0, 20:1, 20:2, 20:4, 22:0 or 22:1, but they are not limited thereto.

The lysophosphatidylethanolamine is a substance produced by decomposition of the second acyl chain in phosphatidylethanolamine, which is one of phospholipids, due to PLA2 enzyme activity. Types thereof may be, for example, p-14:1, 16:0, 18:0, 18:1, 18:2, 20:0, 20:1, 20:2, 20:4, 22:0 or 22:1, but they are not limited thereto.

The metabolic disease is collectively called diseases caused by metabolic disorders in vivo. Specifically, the metabolic disease may be obesity, diabetes, hyperlipidemia, hypertension, hypercholesterolemia, hyperinsulinemia, arteriosclerosis, or fatty liver, but it is not limited thereto.

The term ‘prevention’ refers to any action to inhibit or delay the metabolic disease, and the term ‘treatment’ refers to any action to improve or beneficially change symptoms of an individual suspected of or suffering from the metabolic disease.

The pharmaceutical composition of an embodiment of the present invention may be in the form of a capsule, tablet, granule, injection, ointment, powder or beverage, and may be formulated and used in oral dosage forms such as powder remedies, granules, capsules, tablets, and aqueous suspensions, external preparations, suppositories and injections.

The pharmaceutical composition of an embodiment of the present invention may contain an active ingredient alone, or may further include one or more pharmaceutically acceptable carriers, excipients or diluents.

The pharmaceutical composition of an embodiment of the present invention may include a pharmaceutically acceptable carrier. The pharmaceutically acceptable carrier may be binders, lubricants, disintegrants, excipients, solubilizers, dispersants, stabilizers, suspending agents, coloring agents, flavoring agents, etc. for oral administration. Further, for injections, a buffering agent, a preservative, a soothing agent, solubilizing agent, an isotonic agent, a stabilizer, etc. may be mixed and used. Further, for topical administration, a base agent, an excipient, a lubricant, a preservative, etc. may be used.

The formulation of the pharmaceutical composition of an embodiment of the present invention may be prepared in various ways by mixing the composition with the pharmaceutically acceptable carrier. For example, when administered orally, it may be prepared in the forms of tablets, troches, capsules, elixir, suspension, syrup, wafers, etc. Further, in the case of an injection, it may be prepared in a unit dosage ampoule or a multiple dosage form. In addition, the formulation of the pharmaceutical composition of an embodiment of the present invention may be prepared as a solution, suspension, tablet, capsule, sustained release formulation or the like.

The carriers, excipients and diluents for formulation may include, for example, lactose, dextrose, sucrose, sorbitol, mannitol, xylitol, erythritol, malditol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, mineral oil, filler, anti-aggregative agent, lubricant, wetting agent, fragrance, emulsifier or preservative.

The administration route of the pharmaceutical composition of an embodiment of the present invention may include, for example oral, intravenous, intramuscular, intraarterial, intramedullary, intrathecal, intracardiac, transdermal, subcutaneous, intraperitoneal, intranasal, intestinal, topical, sublingual or rectal, but it is not limited thereto.

The pharmaceutical composition of an embodiment of the present invention may be administered orally or parenterally and, when administered parenterally, external preparations or injection methods such as intraperitoneal, rectal, subcutaneous, intravenous, intramuscular or intrathoracic injection may be selected.

The dosage of the pharmaceutical composition of an embodiment of the present invention may vary depending on the condition and weight of the patient, the degree of the disease, the form of the drug, the route and duration of administration, but may be appropriately selected by those skilled in the art. For example, the pharmaceutical composition may be administered in an administration dosage of 0.0001 to 1000 mg/kg or 0.001 to 500 mg/kg per day, and in terms of administration cycle, may be administered once a day, or may be divided several times. The administration dosage does not limit the scope of the present invention in any way.

In addition, an embodiment of the present invention may provide a health functional food for preventing or improving metabolic diseases, which includes lysophospholipid.

The lysophospholipids and metabolic diseases are as described above.

The health functional food of an embodiment of the present invention may be formulated as one selected from the group consisting of tablets, pills, powder remedies, powders, capsules, and liquid formulations by granules, further adding at least one of carriers, diluents, excipients and additives thereto.

The health functional food may be, for example, various foods, powders, granules, tablets, capsules, syrup, beverages, gums, teas, vitamin complexes, or health functional foods.

Additives that can be included in health functional foods may be selected from the group consisting of natural carbohydrates, flavoring agents, nutrients, vitamins, minerals (electrolytes), flavoring agents (synthetic flavors, natural flavors, etc.), coloring agents, fillers (cheese, chocolate, etc.), pectic acid or salts thereof, alginic acid and salts thereof, organic acids, protective colloidal thickeners, pH adjusters, stabilizers, preservatives, antioxidants, glycerin, alcohols, carbonation agents and pulp.

Examples of natural carbohydrates may be: monosaccharides such as glucose, fructose, and the like; disaccharides such as maltose, sucrose, and the like; and polysaccharides, for example, common sugars such as dextrin and cyclodextrin, and sugar alcohols such as xylitol, sorbitol, and erythritol and the like. Further, as the flavoring agent, natural flavors (thaumatin, stevia extract (e.g., rebaudioside A, glycyrrhizin, etc.)), and synthetic flavors (saccharin, aspartame, etc.) may be advantageously used.

The health functional food may further include various nutrients, vitamins, minerals (electrolytes), flavoring agents such as synthetic flavors and natural flavors, coloring agents and thickeners (cheese, chocolate, etc.), pectic acid and salts thereof, alginic acid and salts thereof, and organic acids, protective colloidal thickeners, pH adjusters, stabilizers, preservatives, glycerin, alcohol, carbonation agents used in carbonated beverages, and pulp for production of natural fruit juices and vegetable beverages and the like.

The carriers, excipients, diluents and additives are not limited thereto, but may be selected from the group consisting of lactose, dextrose, sucrose, sorbitol, mannitol, erythritol, starch, gum acacia, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methyl hydroxy benzoate, propylhydroxy benzoate, talc, magnesium stearate and mineral oil.

When formulating the health functional food of an embodiment of the present invention, it may be prepared using diluents or excipients such as fillers, extenders, binders, wetting agents, disintegrants, and surfactants.

Hereinafter, the present invention will be described in detail by means of examples. The following examples are illustrative of the present invention, therefore the contents of the present invention are not limited to the following examples.

### Example

### 1. Experimental Method

### (1) Cell Culture

C3H10T1/2 cells were purchased from the American Type Culture Collection (ATCC), and in vitro adipocyte experiments were performed using these cells, which were cultured in DMEM (Welgene) supplemented with 10% FBS (Gibco) and 1% P/S at 37° C. in a humidified atmosphere of 5% CO₂. To prepare fully differentiated C3H10T1/2 adipocytes, confluent C3H10T1/2 cells were exposed to BMP4 (R&D system, 20 ng/ml) for 2 days, and induced in a differentiation medium containing IBMX (isobutylmethylxanthine, Cayman, 0.5 mM), dexamethasone (Cayman, 1 μM), insulin (Sigma, 10 μg/mL), indomethacin (Cayman, 0.125 mM), and triiodothyronine (T3, Cayman, 1 nM) for 3 days, then exposed to a maintenance medium containing insulin (1 μg/mL) and T3 (1 nM) for 3 days. To evaluate in vitro effects of lysoplasmalogen, the fully differentiated C3H10T1/2 cells were treated with vehicle control, LPE P-18:0 (1 μM) or LPC P-18:0 (1 μM), LPC P-16:0. (10 μM) or isoproterenol (10 μM) for an indicated time.

### (2) Oxygen Consumption Rate

Oxygen consumption rates (OCRs) were measured with a Seahorse XF Analyzer (Agilent). The differentiated C3H10T1/2 adipocytes (1 μM) treated with LPC P-18:0 (1 μM), LPE P-18:0 or vehicle were washed, and maintained in an assay medium (XF DMEM basal medium, pH 7.4 (Agilent)) supplemented with D-(+)-glucose (25 mM) and L-glutamine (4 mM). The XFp Cell Mito Stress Test Kit (Agilent) was used at concentrations of 2.5 μM oligomycin, 0.5 μM carbonyl cyanide-4-(trifluoromethoxy) phenylhydrazone (FCCP) and 0.5 UM rotenone/antimycin A. Basal and maximal OCRs were calculated by subtracting non-mitochondrial respiration. The ATP production rate was calculated by subtracting the OCR induced by oligomycin A from the basal OCR. Spare respiratory capacity was calculated by subtracting baseline OCR from the maximal OCR. The OCR was normalized to protein concentration. Data were analyzed using Agilent Wave software (version 2.6.0.31).

### (3) Quantification of Intracellular CAMP Level

To measure intracellular CAMP levels in the differentiated C3H10T1/2 adipocytes treated with LPC P-18:0 (1 μM), LPE P-18:0 (1 μM) or isoproterenol (10 μM), The Direct CAMP ELISA Kit (Enzo) was used according to the manufacturer's instructions. C3H10T1/2 adipocytes were incubated along with 0.1 M HCl for 10 minutes, and then scraped, followed by centrifugation. The collected samples and standards added to wells, which are acetylated and coated with goat-rabbit IgG antibody, were incubated along with CAMP conjugated to alkaline phosphatase and rabbit polyclonal antibody against CAMP for 2 hours in a plate shaker at room temperature. After washing three times, p-nitrophenyl phosphate solution was added and incubated at room temperature for 1 hour, then aqueous solution of trisodium phosphate was added thereto. The absorbance of the plate was measured at 405 nm, and the calculated absorbance was converted into pmol/ml of CAMP using AssayFit Pro (v1.31), and normalized to protein concentration measured with the Pierce BCA Protein Assay Kit (Thermo Fisher Scientific).

### (4) Quantitative PCR and Western Blot Analysis

BAT, iWAT and gWAT of mice fed a HFD and administered vehicle control or LPE P-18:0 intraperitoneally were homogenized using the tacoPrep Bead Beater homogenization system in a PROPREP protein extraction solution (iNtRON Biotechnology) containing SIGMAFAST Protease Inhibitor Cocktail (Sigma) and PhoSTOP phosphatase inhibitor (Roche). C3H10T1/2 adipocytes treated with vehicle control, LPE P-18:0 (1 μM) or LPC P-18:0 (1 μM), LPC P-16:0 (10 UM) or isoproterenol (10 μM) were lysed in RIPA Lysis and Extraction Buffer (Thermo Fisher Scientific) containing SIGMAFAST protease inhibitor cocktail (Sigma) and PhoSTOP phosphatase inhibitor (Roche). The extracted protein concentration was quantified by BCA analysis and measured by spectrophotometry at 562 nm (MultiSkan GO, Thermo Fisher Scientific). Proteins were denatured in an SDS sample buffer at 95° C. for 5 min, separated in SDS-PAGE gel, and transferred to a PVDF membrane (Bio-Rad). The membrane was blocked with BSA in 5% non-fat dry milk or TBST (Tris-buffered saline containing 0.1% Tween 20) and incubated overnight along with the primary antibody at 4° C., then cultured with horseradish peroxidase (HRP)-conjugated secondary antibody for 1 hour at room temperature. The primary antibody used for Western blot analysis are listed in Reagents or Resources. Protein expression was detected by a Fusion Solo chemiluminescence imaging system (Vilber Lourmat) and analyzed with EvolutionCapt software (version 17.03). Immunoblots were quantified with National Institutes of Health ImageJ software (version 1.52a).

### (5) Preparation of In Vivo Experimental Mice and Analysis Method

For the diet-induced obesity model, 8-week-old mice were fed a HFD (60% fat, Research Diets, cat #D12492) for 8 weeks. Standardized rodent pellet feed (NCD, Purina, cat #38057) was used as the control feed. Energy expenditure (EE) was estimated by an indirect calorimeter (PhenoMaster, TSE Systems, Germany). Body compositions were measured by nuclear magnetic resonance (NMR) scanning EchoMRI-700 (Echo Medical Systems). To investigate effects of treatment with LPE P-18:0, the mice were fed a HFD for 8 weeks and injected intraperitoneally with LPE P-18:0 (Avanti Polar Lipids, 200 μg/kg/day, cat #852471P) or vehicle for 2 weeks.

### (6) Insulin Tolerance Test (ITT)

For the insulin tolerance test, the mice were fasted for 6 hours before the test, was injected intraperitoneally with insulin (Sigma, 0.75 units/kg body weight, cat #91077C-1G), and blood glucose was measured at a designated time interval. To investigate insulin signaling, the mice were anesthetized, and subjected to injection of insulin (0.75 units/kg body weight) into the inferior vena cava, then adipose tissues thereof were collected 10 minutes after the injection.

### (7) Statistical Analysis

GraphPad Prism 7 software (GraphPad Software, USA) was used for statistical analysis, and statistical significance between two groups was determined by the unpaired t-test. Data are expressed as mean±standard error of the mean (SEM). Comparisons between multiple groups were performed using two-way analysis of variance (ANOVA) along with Bonferroni post hoc analysis to determine P values.

### 2. Experimental Results

### (1) LPE P-18:0 Treatment Promotes Mitochondrial Oxidative Metabolism and Protects Against Diet-Induced Obesity.

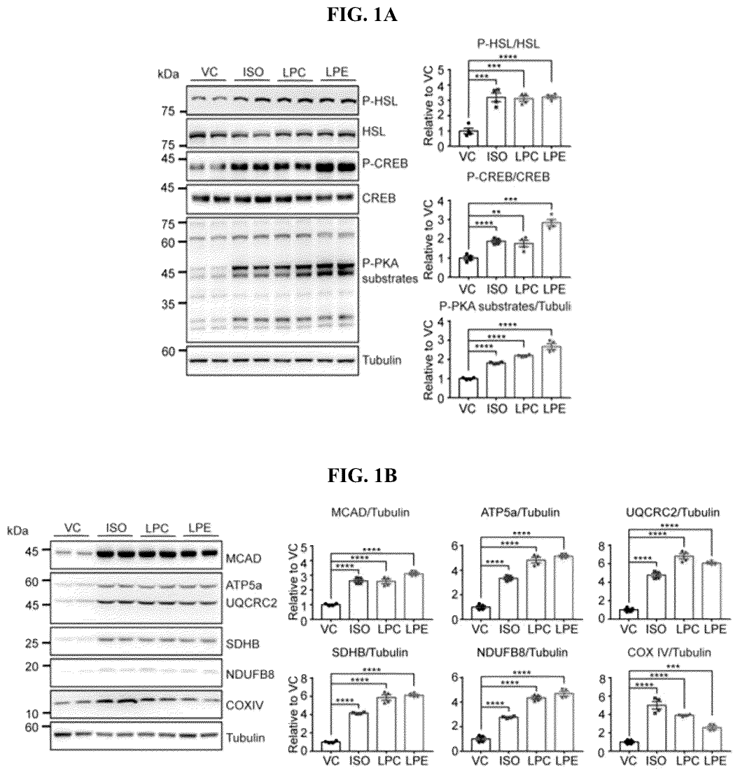

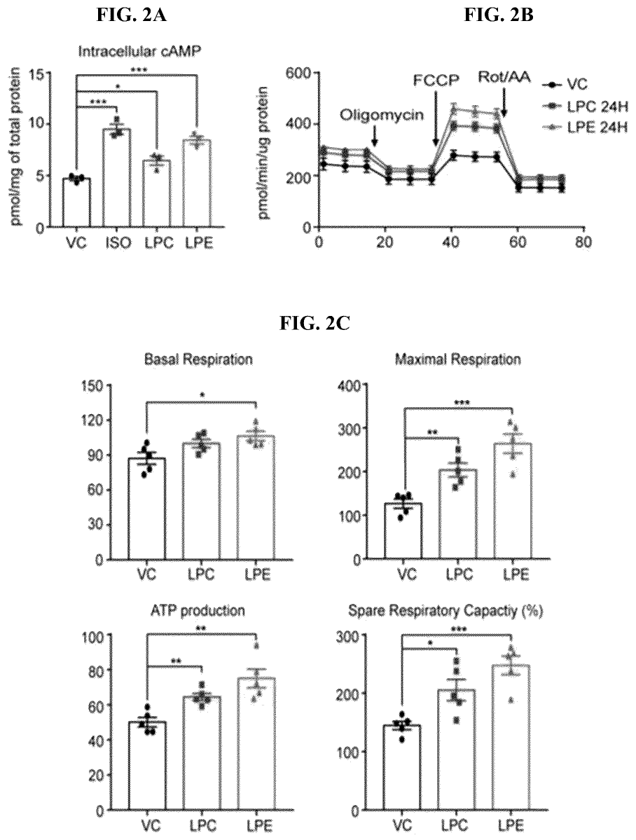

The fully differentiated C3H10T1/2 adipocytes were treated with 1 μM LPC P-18:0 or LPE P-18:0 for 24 or 48 hours. A significant increase in the levels of phosphorylated HSL, CREB and PKA substrates was observed in the LPC P-18:0 and LPE P-18:0 treatment groups (FIG. 1A). Long-term treatment (48 hours) induced significant upregulation of mitochondrial enzymes, including MCAD, ATP5A, UQCRC2, SDHB, NDUFB8, and COXIV (FIG. 1B). The C3H10T1/2 adipocytes treated with LPC P-18:0 or LPE P-18:0 for 24 hours showed an increase in intracellular CAMP levels (FIG. 2A). In addition, treatment with LPC P-18:0 or LPE P-18:0 showed an increase in the maximum respiration rate, ATP production rate and spare respiratory capacity of C3H10T1/2 adipocytes (FIGS. 2B and 2C).

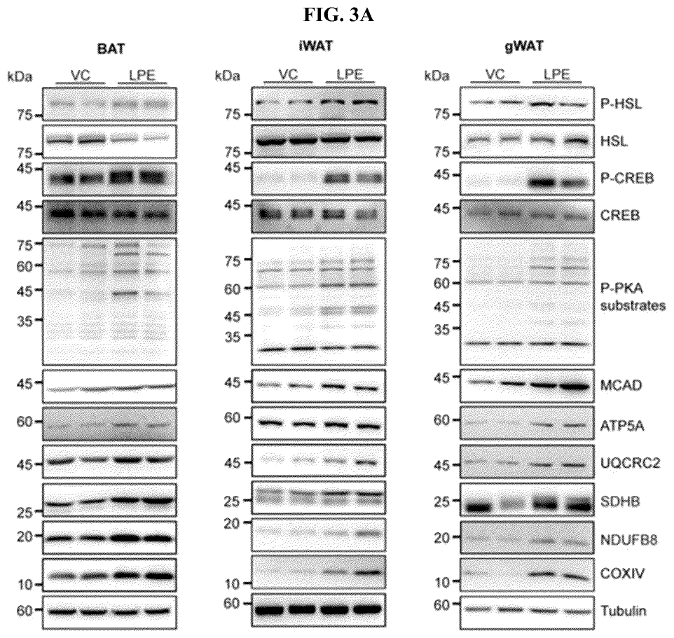

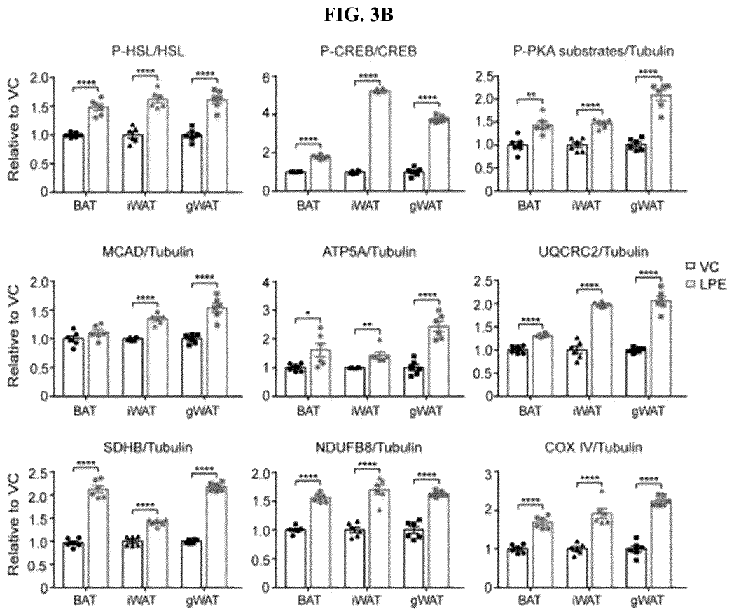

Next, in vivo effects of treatment with LPE P-18:0 were investigated. Immunoblot analysis demonstrated that LPE P-18:0 upregulates PKA signaling and mitochondrial activity and improves glucose tolerance in the adipose tissues (FIGS. 3A to 4).

### (2) LPE P-18:0 Treatment Increases Energy Expenditure and Protects Against Diet-Induced Obesity.

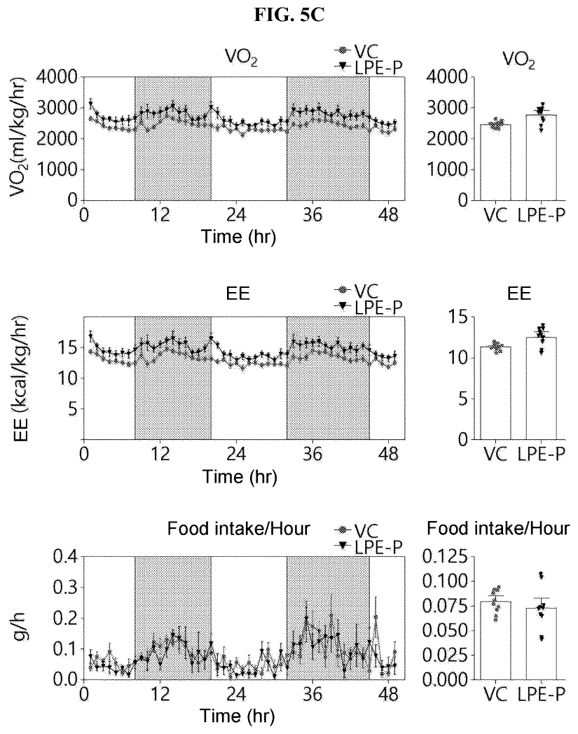

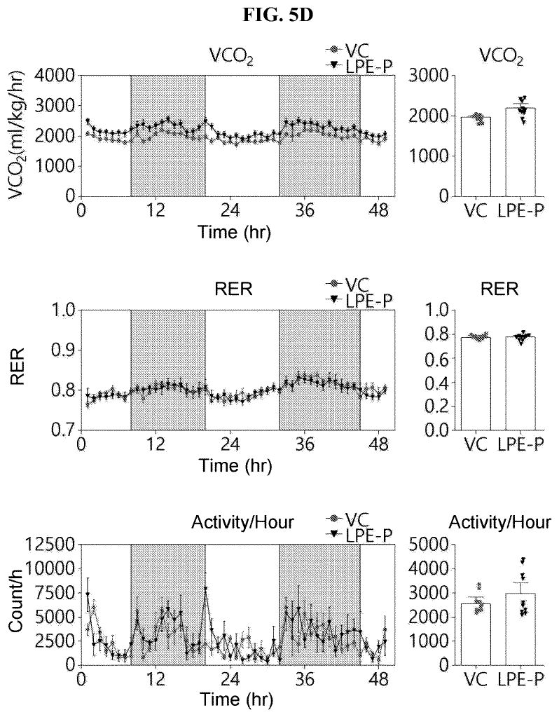

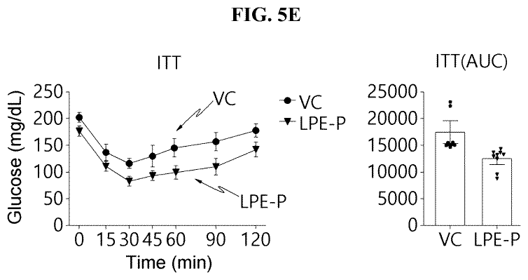

After treatment with LPE P-18:0, the body weight and body fat mass of the mice were decreased. As a result of indirect calorimetry analysis, VO₂ and energy expenditure were increased. Food intake was decreased, but activity was increased. As a result of intraperitoneal insulin tolerance test, insulin tolerance was decreased (FIGS. 5A to 5E).

### (3) LPC P-16:0 Treatment Activates PKA Downstream Signaling.

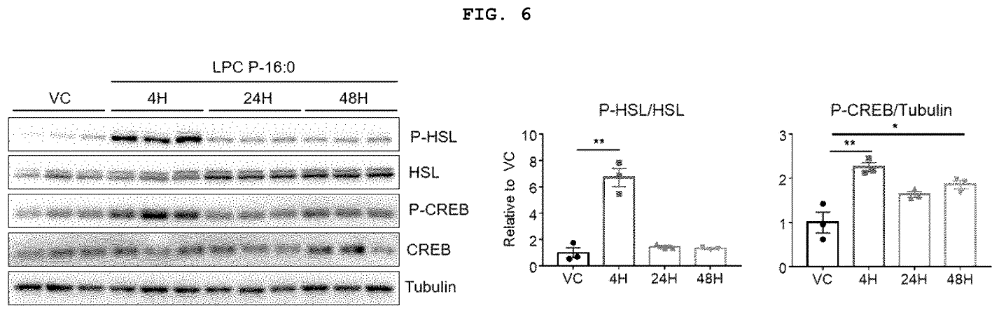

In addition to LPC P-18:0 and LPE P-18:0, effects of treatment with lysophospholipid LPC P-16:0 were investigated. The fully differentiated C3H10T1/2 adipocytes were treated with 10 μM LPC P-16:0 for 4, 24 or 48 hours, and vehicle (4 mg/mL fatty acid-free BSA) was used as the control. A significant increase in the levels of phosphorylated HSL and CREB was observed in the group treated with LPC P-16:0 for 4 hours. This result indicates that lipolysis could occur in a PKA-dependent manner due to LPC P-16:0 (FIG. 6).

1-8. (canceled)

9. A method for treating a metabolic disease, the method comprising administering a composition comprising lysophospholipid (LPL) to a subject in need thereof.

10. The method of claim 9, wherein the metabolic disease is selected from the group consisting of obesity, diabetes, hyperlipidemia, hypertension, hypercholesterolemia, hyperinsulinemia, arteriosclerosis, fatty liver, and a combination thereof.

11. The method of claim 9, wherein the lysophospholipid is lysophosphatidyl choline (LPC) and/or lysophosphatidyl ethanolamine (LPE).

12. The method of claim 11, wherein the lysophosphatidylcholine and/or the lysophosphatidylethanolamine are at least one selected from the group consisting of p-14:1, 16:0, 18:0, 18:1, 20:0, 20:2, 20:4, 22:5 and 22:6.

13. The method of claim 9, wherein the metabolic disease is obesity.

14. The method of claim 9, wherein the metabolic disease is diabetes.

15. The method of claim 9, wherein the metabolic disease is hyperlipidemia.

16. The method of claim 9, wherein the metabolic disease is hypertension.

17. The method of claim 9, wherein the metabolic disease is hypercholesterolemia.

18. The method of claim 9, wherein the metabolic disease is hyperinsulinemia.

19. The method of claim 9, wherein the metabolic disease is arteriosclerosis.

20. The method of claim 9, wherein the metabolic disease is fatty liver.

21. The method of claim 9, wherein the composition is a pharmaceutical composition comprising the lysophospholipid (LPL) and at least one of a pharmaceutically acceptable carrier, an excipient, or a diluent.

22. The method of claim 9, wherein the composition is included in a health functional food.
